(19) United States
(12) Patent Application Publication
(10) Pub. No.: US 2025/0388631 A1
(43) Pub. Date: Dec. 25, 2025

(54) RECOMBINANT H-FERRITIN PROTEINS FOR IRON DEFICIENCY, IRON TOXICITY AND CHEMOTHERAPY

(71) Applicant: The Penn State Research Foundation, University Park, PA (US)

(72) Inventors: Kondaiah Palsa, Hershey, PA (US); Elizabeth B. Neely, Hershey, PA (US); James R. Connor, Hershey, PA (US)

(21) Appl. No.: 19/234,707

(22) Filed: Jun. 11, 2025

Related U.S. Application Data
(60) Provisional application No. 63/661,667, filed on Jun. 19, 2024.

(51) Int. Cl. C07K 14/435; A61K 38/00; A61P 7/06

(52) U.S. Cl. CPC C07K 14/435; A61P 7/06; A61K 38/00

(57) ABSTRACT

The present disclosure includes compositions possessing a recombinant H-ferritin homopolymer that has a plurality of H-ferritin subunits with an amino acid sequence of SEQ ID NO: 1. Methods for treating an iron-related condition in a subject and for treating a neurological or blood disorder in a subject with such compositions are also disclosed.

### CROSS-REFERENCE TO RELATED APPLICATIONS

This application claims the benefit of priority of U.S. Provisional Application Ser. No. 63/661,667, filed on Jun. 19, 2024, and entitled “RECOMBINANT H-FERRITIN PROTEINS FOR IRON DEFICIENCY, IRON TOXICITY AND CHEMOTHERAPY”, the entirety of which is incorporated herein by reference.

### STATEMENT OF GOVERNMENT SUPPORT

This invention was made with government support under Grant No. NS113912 awarded by the National Institutes of Health. The Government has certain rights in the invention.

### FIELD OF INVENTION

The present disclosure relates to compositions containing H-ferritin proteins and uses of such compositions to treat iron deficiency, iron toxicity, and neurological/blood disorder in subjects in need thereof.

### REFERENCE TO SEQUENCE LISTING

This specification includes a sequence listing submitted herewith, which includes the file entitled 0073605-000987.xml having the following size: 1,917 bytes which was created Mar. 12, 2025, the contents of which are incorporated by reference herein.

### BACKGROUND

Iron is an essential mineral for growth and development. For example, the human body uses iron to make hemoglobin, a protein in red blood cells that carries oxygen from the lungs to all parts of the body, and myoglobin, a protein that provides oxygen to muscles. The human body also needs iron as a cofactor for some proteins.

Iron is also essential for proper functioning of the central nervous system through its role in brain growth, myelin formation, and the synthesis of neurotransmitters (see Beard J L, Connor J R, Jones B C, “Iron in the brain”, Nutr. Rev., 2009; 51(6):157-170 and Beard J L, Connor J R, “Iron status and neural functioning”, Annu. Rev. Nutr., 2003; 23:41-58). Iron uptake in brain has been shown to be age- and sex-dependent (see Chiou B, Neely E B, Mcdevitt D S, Simpson I A, Connor J R, “Transferrin and H-ferritin involvement in brain iron acquisition during postnatal development: impact of sex and genotype”, J Neurochem., 2020; 152(3):381-396; Duck K A, Neely E B, Simpson I A, Connor J R, “A role for sex and a common HFE gene variant in brain iron uptake”, J Cereb. Blood Flow Metab., 2018; 38(3):540-548; Castner S A, Xiao L, Becker J B, “Sex differences in striatal dopamine: in vivo microdialysis and behavioral studies”, Brain Res., 1993; 610(1):127-134; and Yang S, Li C, Zhang W, Wang W, Tang Y, “Sex differences in the white matter and myelinated nerve fibers of long-Evans rats”, Brain Res., 2008; 1216:16-23).

Iron deficiency (ID) anemia, especially during development, can critically impact neurological and cognitive function to the extent that the deficits persist into adulthood (see Beard J, “Iron deficiency alters brain development and functioning”, J Nutr., 2003; 133(5):14685-14725; and Georgieff M K, “Long-term brain and behavioral consequences of early iron deficiency”, Nutr. Rev., 2011; 69(Suppl 1): S43-S48). In adults, ID can also alter cognitive function (see Bruner A B, Joffe A, Duggan A K, Casella J F, Brandt J, “Randomised study of cognitive effects of iron supplementation in non-anemic iron-deficient adolescent girls”, Lancet, 1996; 348(9033):992-996), but few studies have been carried out to confirm if ID throughout nondevelopment periods of life is correlated with changes in behavior, cognition, brain function, and responses to iron therapy.

### SUMMARY OF THE DISCLOSURE

Disclosed herein is a composition including (i.e., comprising) a recombinant H-ferritin homopolymer including one or more H-ferritin subunits, wherein at least one H-ferritin subunit has an amino acid sequence of SEQ ID NO: 1. In some embodiments, the recombinant H-ferritin homopolymer includes at least 24 H-ferritin subunits. In some embodiments, the recombinant H-ferritin homopolymer includes 24 H-ferritin subunits.

Also disclosed herein is a genetically engineered microbe or host cell that produces the recombinant H-ferritin homopolymer described herein.

Also disclosed herein is a method for treating iron-related conditions in a subject, the method including administering to the subject a composition including i) a recombinant H-ferritin homopolymer having a plurality of H-ferritin subunits, wherein at least one H-ferritin subunit has an amino acid sequence of SEQ ID NO: 1, and ii) an iron element stored within the recombinant H-ferritin homopolymer. In some embodiments, the recombinant H-ferritin homopolymer includes at least 24 H-ferritin subunits. In some embodiments, the recombinant H-ferritin homopolymer includes 24 H-ferritin subunits. In some embodiments, the recombinant H-ferritin homopolymer includes a holo-recombinant H-ferritin homopolymer. In some embodiments, when compared to human H-ferritin, the recombinant H-ferritin homopolymer can include one or more histidine (His) tags in its sequence.

Also disclosed herein is a method for treating a neurological or blood disorder in a subject, the method including administering to the subject a composition including a recombinant H-ferritin homopolymer having one or more H-ferritin subunits, wherein at least a H-ferritin subunit has an amino acid sequence of SEQ ID NO: 1. In some embodiments, the recombinant H-ferritin homopolymer includes at least 24 H-ferritin subunits. In some embodiments, the recombinant H-ferritin homopolymer includes 24 H-ferritin subunits.

This disclosure provides a model for addressing the public health debate over whether to provide iron supplementation to children and adults suffering from ID, iron accumulation, or other iron-related conditions, along with methods for using H-ferritin (FTH1) to treat ID, excess iron accumulation, or other iron-related conditions.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings.

Other features and advantages of the compositions and methods disclosed herein will be apparent to those skilled in the art upon reading the following detailed description in conjugation with the exemplary embodiments illustrated in the drawings.

FIGS. 1A-1D depict the results from an iron uptake experiment involving an exemplary composition containing a recombinant H-ferritin homopolymer (FTH1) disclosed herein and developmental mouse models of ID. FIG. 1A depicts the two experimental mice groups, i.e., the control group and the ID group, used in the study. FIG. 1B compares the brain uptake of ⁵⁵Fe-bound recombinant H-ferritin homopolymer (⁵⁵Fe-FTH1), between the control and ID mice groups, in both developing male and female mice. FIG. 1C compares the brain uptake of ⁵⁵Fe-bound transferrin (⁵⁵Fe-Tf), between the control and ID mice groups, in both developing male and female mice. FIG. 1D compares the relative brain uptake of ⁵⁵Fe bound to the recombinant H-ferritin homopolymer (FTH1) and to the transferrin (Tf), respectively, in both developing male and female mice with ID, indicating a superior ⁵⁵Fe uptake efficiency for FTH1.

FIGS. 2A-2C depict the results from an iron uptake experiment involving an exemplary composition containing a recombinant H-ferritin homopolymer disclosed herein and adult mouse models of ID. FIG. 2A compares the brain uptake of the ⁵⁵Fe-bound recombinant H-ferritin homopolymer (⁵⁵Fe-FTH1), between the control and ID mice groups, in both adult male and female mice. FIG. 2B compares the brain uptake of ⁵⁵Fe-bound transferrin (⁵⁵Fe-Tf), between the control and ID mice groups, in both adult male and female mice. FIG. 2C compares the relative brain uptake of ⁵⁵Fe bound to the recombinant H-ferritin homopolymer (FTH1) and the transferrin (Tf), respectively, in both adult male and female mice.

FIGS. 3A-3D depict the results of an iron uptake experiment involving an exemplary composition containing a recombinant H-ferritin homopolymer disclosed herein and two groups of mouse models on a control diet and an iron-deficient diet, respectively. Specifically, iron uptakes between postnatal day 15 (PND15) and postnatal day 85 (PND85) were compared. FIG. 3A compares the brain uptake of the ⁵⁵Fe-bound recombinant H-ferritin homopolymer (⁵⁵Fe-FTH1) on PND15 and PND 85, in both developing and adult male and female mouse models on a control diet. FIG. 3B compares the brain uptake of ⁵⁵Fe-bound transferrin (⁵⁵Fe-Tf) on PND15 and PND 85, in both developing and adult male and female mouse models on a control diet. FIG. 3C compares the brain uptake of the ⁵⁵Fe-bound recombinant H-ferritin homopolymer (⁵⁵Fe-FTH1) on PND15 and PND 85, in both developing and adult male and female mouse models on an iron-deficient diet. FIG. 3D compares the brain uptake of ⁵⁵Fe-bound transferrin (⁵⁵Fe-Tf) on PND15 and PND 85, in both developing and adult male and female mouse models on an iron-deficient diet.

FIGS. 4A-4C depict the results of an iron redistribution experiment involving an exemplary composition containing a recombinant apo-H-ferritin homopolymer disclosed herein. FIG. 4A depicts the hemoglobin (Hb) levels in a transferrin-deficient mouse model after seven days of treatment with PBS (hpx), PBS and transferrin (hpx+Tf), and PBS and the recombinant apo-H-ferritin homopolymer (hpx+FTH1), respectively; while the treatment using Tf significantly increased the Hb levels, the treatment using FTH1 did not result in such an increase. FIG. 4B depicts the hematocrit (HCT) levels in a transferrin-deficient mouse model after seven days of treatment with PBS (hpx), PBS and transferrin (hpx+Tf), and PBS and the recombinant apo-H-ferritin homopolymer (hpx+FTH1), respectively; while the treatment using Tf significantly increased the HCT levels, the treatment using FTH1 did not result in such an increase. FIG. 4C depicts the serum iron levels in a transferrin-deficient mouse model after seven days of treatment with PBS (hpx), PBS and transferrin (hpx+Tf), and PBS and the recombinant apo-H-ferritin homopolymer (hpx+FTH1), respectively; both Tf and FTH1 decreased the serum iron levels.

FIG. 5 depicts the liver iron levels in a transferrin-deficient mouse model after treatment with PBS (hpx), PBS and transferrin (hpx+Tf), and PBS and an exemplary recombinant apo-H-ferritin homopolymer (hpx+FTH1), respectively; both Tf and FTH1 significantly decreased the liver iron levels.

FIG. 6 depicts the spleen iron levels in a transferrin-deficient mouse model after treatment with PBS (hpx), PBS and transferrin (hpx+Tf), and PBS and an exemplary recombinant apo-H-ferritin homopolymer (hpx+FTH1), respectively; both Tf and FTH1 significantly increased the spleen iron levels.

FIG. 7 depicts the brain iron levels in a transferrin-deficient mouse model after treatment with PBS (hpx), PBS and transferrin (hpx+Tf), and PBS and an exemplary recombinant apo-H-ferritin homopolymer (hpx+FTH1), respectively.

The drawings are not necessarily to scale and may be illustrated by phantom lines, diagrammatic representations, and fragmentary views. In certain instances, details that are not necessary for an understanding of the embodiments or that render other details difficult to perceive may have been omitted.

### DETAILED DESCRIPTION

To facilitate the understanding of this invention, a number of terms are defined below and throughout the disclosure. Unless otherwise defined, all technical and scientific terms used herein have the same meaning as commonly understood by one of ordinary skill in the art to which this invention belongs. In case of conflict, the present specification, including definitions, will control. The terminology herein is used to describe specific embodiments of the invention, but their usage does not limit the invention, except as outlined in the claims. All publications, patent applications, patents, and other references mentioned herein are incorporated by reference in their entirety.

It is to be understood that any aspect and/or element of any embodiment of the method(s) described herein or otherwise may be combined in any way to form additional embodiments of the method(s), all of which are within the scope of the method(s).

As used herein, including the claims, the phrase “at least some” means “one or more” and includes the case of only one. Thus, e.g., the phrase “at least some ABCs” means “one or more ABCs” and includes the case of only one ABC.

As used herein, including the claims, the term “at least one” should be understood as meaning “one or more” and therefore includes both embodiments that include one or multiple components. Furthermore, dependent claims that refer to independent claims that describe features with “at least one” have the same meaning, both when the feature is referred to as “the” and “the at least one”.

As used herein, the term “portion” means some or all. Therefore, for example, “a portion of X” may include some of “X” or all of “X”. In the context of a conversation, the term “portion” means some or all of the conversation.

As used herein, including the claims, the phrase “using” means “using at least” and is not exclusive. Thus, e.g., the phrase “using X” means “using at least X”. Unless specifically stated by use of the word “only”, the phrase “using X” does not mean “using only X”.

As used herein, including the claims, the phrase “based on” means “based in part on” or “based, at least in part, on” and is not exclusive. Thus, e.g., the phrase “based on factor X” means “based in part on factor X” or “based, at least in part, on factor X”. Unless specifically stated by use of the word “only”, the phrase “based on X” does not mean “based only on X”.

In general, as used herein, including the claims, unless the word “only” is specifically used in a phrase, it should not be read into that phrase.

As used herein, including the claims, the terms “multiple” and “plurality” mean “two or more,” and include the case of “two”. Thus, e.g., the phrase “multiple ABCs” means “two or more ABCs” and includes “two ABCs”. Similarly, e.g., the phrase “multiple PQRs” means “two or more PQRs” and includes “two PQRs”.

The present invention also covers the exact terms, features, values, and ranges, etc., in case these terms, features, values, and ranges, etc., are used in conjunction with terms such as “about”, “around”, “generally”, “substantially”, “essentially”, “at least”, etc. Thus, e.g., “about 3” or “approximately 3” shall also cover exactly 3, and “substantially constant” shall also cover exactly constant.

As used herein, unless stated otherwise, the terms “about” or “approximately” refer to a value that is within 5% above or below the value being described. In addition, it is understood that reference to a range of a first value to a second value includes the range of the stated values, e.g., a range of about 1 to about 5 also includes the more precise range of 1 to 5. It is also understood that the ranges disclosed herein include any selected subrange within the stated range, e.g., a subrange of about 50 to about 60 is contemplated in a disclosed range of about 1 to about 100.

As used herein, including the claims, singular forms of terms are to be construed as also including the plural form and vice versa, unless the context indicates otherwise. Thus, it should be noted that as used herein, the singular forms “a”, “an”, and “the” include plural references unless the context clearly dictates otherwise. In other words, terms such as “a”, “an”, and “the” are not intended to refer to only a singular entity but include the general class of which a specific example may be used for illustration.

Throughout the description and claims, the terms “comprise”, “including”, “having”, “contain”, and their variations should be understood as meaning “including but not limited to” and are not intended to exclude other components unless specifically so stated.

As used herein, the terms “administration” or “administering” refer to a method of giving a dosage of a compound or pharmaceutical composition to a subject. A composition described herein may be administered to a subject by any one of a variety of manners or a combination of varieties of manners. For example, a composition may be administered orally, nasally, intraperitoneally, or parenterally, by intravenous, intramuscular, topical, or subcutaneous routes, or by injection into tissue, consistent with details described elsewhere in this disclosure.

As used herein, an “effective amount” or “therapeutically effective amount” is the amount of a composition of this disclosure which, when administered to a subject, is sufficient to effect treatment of a disease or condition in the subject. The amount of a composition of this disclosure which constitutes a “therapeutically effective amount” may vary depending on the composition, the condition and its severity, the manner of administration, and the age of the subject to be treated.

As used herein, the terms “treat”, “treating”, or “treatment” refer to administration of a compound or pharmaceutical composition for a therapeutic purpose. To “treat a disorder” or use for “therapeutic treatment” refers to administering treatment to a patient already suffering from a disease to ameliorate the disease or one or more symptoms thereof to improve the patient's condition (e.g., by reducing one or more symptoms of a neurological disorder). The term “therapeutic” includes the effect of mitigating deleterious clinical effects of certain processes (i.e., consequences of the process, rather than the symptoms of processes). As nonlimiting examples, a treatment may include (i) preventing a disease or condition from occurring in a subject, in particular, when such subject is predisposed to the condition but has not yet been diagnosed as having it; (ii) inhibiting a disease or condition, i.e., arresting its development; (iii) relieving a disease or condition, i.e., causing regression of the disease or condition; or (iv) relieving the symptoms resulting from a disease or condition, i.e., relieving pain without addressing the underlying disease or condition.

It will be appreciated that variations to the embodiments of the invention can be made while still falling within the scope of the invention. Alternative features serving the same, equivalent, or similar purpose can replace features disclosed in the specification, unless stated otherwise. Thus, unless stated otherwise, each feature disclosed represents one example of a generic series of equivalent or similar features.

Use of exemplary language, such as “for instance”, “such as”, “for example” (“e.g.,”), and the like, is merely intended to better illustrate the invention and does not indicate a limitation on the scope of the invention unless specifically so claimed.

While the invention has been described in connection with what is presently considered to be the most practical and embodiments thereof are further described in the examples below, it is to be understood that the invention is not to be limited to the disclosed embodiment but is intended to cover various modifications and equivalent arrangements included within the spirit and scope of the appended claims.

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, and/or components have not been described in detail in order to avoid unnecessarily obscuring claimed subject matter.

Iron is an essential mineral for growth and development. Dietary iron enters the blood via transport through enterocytes and is released as ferric iron (see Sharp P, Srai S K, “Molecular mechanisms involved in intestinal iron absorption”, World J. Gastroenterol., 2007; 13(35):4716-4724). In the blood, apo-transferrin binds to the ferric iron and delivers it to the targeted tissues as transferrin-bound iron (see Kondaiah P, Yaduvanshi P S, Sharp P A, Pullakhandam R, “Iron and zinc homeostasis and interactions: does enteric zinc excretion cross-talk with intestinal iron absorption?”, Nutrients, 2019; 11(8):1885; and Knutson M D, “Iron transport proteins: gateways of cellular and systemic iron homeostasis”, J. Biol. Chem., 2017; 292(31):12735-12743). Studies on iron transport in brain have traditionally focused on transferrin-bound iron as the classical iron transporter to the brain (see Gkouvatsos K, Papanikolaou G, Pantopoulos K, “Regulation of iron transport and the role of transferrin”, Biochim. Biophys. Acta. (BBA)—Gen. Sub., 2012; 1820(3):188-202, 14). Transferrin-bound iron binds to the transferrin receptor 1 (TfR1) on the apical side of endothelial cells in the blood-brain barrier and is endocytosed. Iron is transported to the brain parenchyma as a free iron via the iron exporter ferroportin1/hephaestin (FPN1/HEPH) (see Simpson I A, Ponnuru P, Klinger M E, “A novel model for brain iron uptake: introducing the concept of regulation”, J. Cereb. Blood Flow Metab., 2015; 35(1):48-57) or as a Tf-iron complex (see Palsa K, Baringer S L, Shenoy G, Simpson I A, Connor J R, “Exosomes are involved in iron transport from human blood-brain barrier endothelial cells and are modified by endothelial cell iron status”, J. Biol. Chem., 2023; 299:102868; and Chiou B, Neal E H, Bowman A B, Lippmann E S, Simpson I A, Connor J R, “Endothelial cells are critical regulators of iron transport in a model of the human blood-brain barrier”, J. Cereb. Blood Flow Metab., 2019; 39(11):2117-2131).

H-ferritin (FTH1) can also transport iron to the brain (see Palsa K, Baringer S L, Shenoy G, Simpson I A, Connor J R, “Exosomes are involved in iron transport from human blood-brain barrier endothelial cells and are modified by endothelial cell iron status”, J. Biol. Chem., 2023; 299:102868; Fisher J, Devraj K, Ingram J, “Ferritin: a novel mechanism for delivery of iron to the brain and other organs”, Am. J. Physiol. Cell Physiol., 2007; 293(2):C641-C649; and Baringer S L, Neely E B, Palsa K, Simpson I A, Connor J R, “Regulation of brain iron uptake by apo- and holo-transferrin is dependent on sex and delivery protein”, Fluids Barriers CNS., 2022; 19(1):49). FTH1 is a type of ferritin, a cellular protein that stores iron (approximately 2000 iron atoms) and typically consists of 24 subunits with different ratios of H- and L-ferritin subunits that are dependent upon the organ and cell (see Arosio P, Ingrassia R, Cavadini P, “Ferritins: a family of molecules for iron storage, antioxidation and more”, Biochim. Biophys. Acta. Gen. Subj., 2009; 1790(7):589-599; and Harrison P M, Arosio P, “The ferritins: molecular properties, iron storage function and cellular regulation”, Biochim. Biophys. Acta. Bioenerg., 1996; 1275(3):161-203). For example, in the brain, FTH1 is found mainly in neurons, whereas L-ferritin is the predominant form in microglial cells. Oligodendrocytes and astrocytes contain both H and L subunits (see Connor J R, Boeshore K L, Benkovic S A, Menzies S L, “Isoforms of ferritin have a specific cellular distribution in the brain”, J. Neurosci. Res., 1994; 37(4):461-465; and Han J, Beard J L, Day J R, Connor J R, “H and L ferritin subunit mRNA expression differs in brains of control and iron-deficient rats”, J. Nutr., 2002; 132(9):2769-2774).

Though transferrin-bound iron and FTH1-bound iron can transport iron to the brain, there is still a considerable public health debate as to whether or not to provide iron supplementation to children and even adults suffering from ID due to the lack of knowledge regarding iron regulation and uptake into the brain. Furthermore, though iron is an essential nutrient for growth and development, it can lead to toxic side effects if it accumulates in the body in excess levels. For example, dietary iron enters the blood via transport through enterocytes and is released as ferric iron. In the blood, apo-transferrin binds to the ferric iron and delivers it to the targeted tissues as transferrin-bound iron (see Coffey R, Ganz T, “Iron homeostasis: an anthropocentric perspective”, J. Biol. Chem., 2017; 292(31):12727-12734; and Kondaiah P, Yaduvanshi P S, Sharp P A, Pullakhandam R, “Iron and zinc homeostasis and interactions: does enteric zinc excretion cross-talk with intestinal iron absorption?”, Nutrients, 2019; 11(8):1885). While body iron levels are determined in theory by a balance between absorption and excretion, the current paradigm in the field of iron biology states that the rate of absorption determines iron levels. Iron absorption is regulated prominently by hepcidin, a hormone synthesized mainly by the liver that inhibits iron export from duodenal enterocytes and other types of cells. Hepcidin expression is suppressed by anemia, leading to increased iron absorption; hepcidin expression is stimulated by iron excess and inflammation, leading to decreased absorption (see Wallace D F, “The regulation of iron absorption and homeostasis”, Clin. Biochem. Rev., 2016; 37(2):51-62; and Kawabata H, “The mechanisms of systemic iron homeostasis and etiology, diagnosis, and treatment of hereditary hemochromatosis”, Int. J. Hematol., 2018; 107(1):31-43). Hepcidin deficiency is central to common inherited diseases of iron excess such as hereditary hemochromatosis and β-thalassemia (see Brissot P, Cavey T, Ropert M, Guggenbuhl P, Loréal O, “Genetic hemochromatosis: pathophysiology, diagnostic and therapeutic management”, Presse. Med., 2017; 46(12 Pt 2):e288-e295; Asadov C, Alimirzoeva Z, Mammadova T, Aliyeva G, Gafarova S, Mammadov J, “β-thalassemia intermedia: a comprehensive overview and novel approaches”, Int. J. Hematol., 2018; 108(1):5-21; and Gupta R, Musallam K M, Taher A T, Rivella S, “Ineffective erythropoiesis: anemia and iron overload”, Hematol. Oncol. Clin. North Am., 2018; 32(2):213-221).

One aspect of the present disclosure is a composition including a recombinant H-ferritin homopolymer. The recombinant H-ferritin homopolymer includes a plurality of H-ferritin subunits, wherein at least one H-ferritin subunit has an amino acid sequence of

(SEQ ID NO: 1)
MTTASTSQVRQNYHQDSEAAINRQINLELYASYVYLSMSYYFDRDDVAL
KNFAKYFLHQSHEEREHAEKLMKLQNQRGGRIFLQDIKKPDCDDWESGL
NAMECALHLEKNVNQSLLEFPSPISPSPSCWHHYTTNRPQPQHHLLRPR
RRKRPHSIPTPILIFRSP.

In some embodiments, the recombinant H-ferritin homopolymer can contain at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, or at least 24 H-ferritin subunits.

In some embodiments, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, or at least 24 H-ferritin subunits of the recombinant H-ferritin homopolymer have an amino acid sequence of SEQ ID NO: 1.

In some embodiments, at least one H-ferritin subunit of the recombinant H-ferritin homopolymer has a molecular weight of about 21 kDa.

In some embodiments, the recombinant H-ferritin homopolymer has a molecular weight of about 50 kDa, about 100 kDa, about 200 kDa, about 300 kDa, about 400 kDa, about 500 kDa, about 600 kDa, about 700 kDa, about 800 kDa, about 900 kDa, or about 1,000 kDa. In some embodiments, the recombinant H-ferritin homopolymer has a molecular weight of about 500 kDa.

In some embodiments, the recombinant H-ferritin homopolymer has a molecular weight of at least 50 kDa, at least 100 kDa, at least 200 kDa, at least 300 kDa, at least 400 kDa, at least 500 kDa, at least 600 kDa, at least 700 kDa, at least 800 kDa, at least 900 kDa, at least 1,000 kDa, etc. For reference, human ferritin is a 24-subunit, 480-kDa, 12-nm globular protein.

As used herein, a “ferritin” is a type of blood protein that is capable of containing or storing iron. Ferritin plays an important role in iron storage and iron homeostasis in the human body, making iron available for critical cellular processes while protecting lipids, DNA, and other proteins from potentially toxic effects of iron (see Knovich et al., “Ferritin for the clinician”, Blood Rev., 2009; 23(3):495-104). A ferritin can contain two types of subunits: ferritin heavy chain (i.e., H-ferritin subunit) and ferritin light chain (i.e., L-ferritin subunit). Such designations of heavy and light are a modern recasting of designations that originally reflected the organs from which the two forms were isolated: “H” for heart and “L” for liver, respectively. As a nonlimiting example, an H-ferritin subunit can contain 182 amino acids with a molecular weight of 21,000 Da, whereas an L-ferritin subunit can contain 174 amino acids and have a molecular weight of 18,500 Da; the two types of ferritin subunits can have ˜53% protein sequence identity. The H subunit can be responsible for catalyzing the oxidation of iron(II), whereas the L subunit can host the site of nucleation and storage of iron. The ratio of these two subunits can vary depending on the tissue type at which the ferritin is synthesized and can be modified in certain inflammatory and infectious conditions. As a nonlimiting example, the ratio of H:L will be higher in tissues where iron oxidation activity is high and iron detoxification is needed, such as the heart or the brain, whereas tissues such as the spleen are used more for storage and will accordingly have a lower H:L ratio instead. As another nonlimiting example, the human liver produces ferritin that is 50% H and 50% L.

As used herein, a “holo-ferritin” is a type of ferritin that includes one or more iron species. The terms ferritin and holo-ferritin may be used interchangeably throughout this disclosure. In contrast, as used herein, an “apo-ferritin” is a type of ferritin that is iron-free.

A ferritin can contain a cavity. As a nonlimiting example, human ferritin contains a cavity with a diameter of 8 nm. A ferritin can also have a ferroxidase activity. As used herein, a “ferroxidase” is an enzyme that catalyzes and accelerates the conversion from Fe²⁺ to Fe³⁺. Upon such oxidation, Fe³⁺ can be internalized and sequestered in the cavity of ferritin. For reference, a 24-subunit human ferritin protein can store up to 4500 iron ions in its cavity, which accounts for up to 24% of the ferritin's weight.

As used herein, a “H-ferritin homopolymer” is a homopolymer that includes a plurality of H-ferritin subunits. As a nonlimiting example, each H-ferritin subunit of the plurality of H-ferritin subunits can have an amino acid sequence according to SEQ ID NO: 1. As another nonlimiting example, at least one H-ferritin subunit of the plurality of H-ferritin subunits can have an amino acid sequence according to SEQ ID NO: 1. As another nonlimiting example, the plurality (e.g., 24) of H-ferritin subunits can undergo a spontaneous (i.e., organic) self-assembly process to form the H-ferritin homopolymer; this process can occur in the cytosol of a genetically engineered microbe, such as without limitation a genetically engineered E. coli strain. As used herein, a “homopolymer” is a polymer made of identical or substantially identical monomer units that are linked or assembled together via chemical bonds and/or intermolecular forces. As used herein, a “polymer” is a chemical species, or an assembly of chemical species, with a structure that includes a plurality of monomer units that are typically covalently linked with one another. As nonlimiting examples, a polymer may include a biopolymer such as without limitation a DNA, an RNA, an oligonucleotide, a peptide, or a protein.

In some embodiments, the recombinant H-ferritin homopolymer includes an apo-recombinant H-ferritin homopolymer. As used herein, an “apo-recombinant H-ferritin homopolymer” or “apo-FTH1” is a recombinant, H-ferritin homopolymer with no iron bound thereto or stored therein. In other words, similar to an apo-ferritin, an apo-recombinant H-ferritin homopolymer is a recombinant H-ferritin homopolymer that is iron-free. The apo-FTH1 described herein is capable of reducing iron overload and enhancing redistribution of iron, e.g., by functioning as an ionophore. In some embodiments, the recombinant H-ferritin homopolymer includes a holo-recombinant H-ferritin homopolymer. As used herein, a “holo-recombinant H-ferritin homopolymer” is a recombinant H-ferritin homopolymer with one or more iron species bound thereto or stored therein.

As used herein, a “recombinant” H-ferritin homopolymer is a type of H-ferritin homopolymer prepared using recombinant DNA technology. As used herein, “recombinant DNA technology”, often referred to as genetic engineering, is a type of technology that involves using enzymes and various laboratory techniques to manipulate and isolate DNA segments of interest. Recombinant DNA technology can be used to combine (or splice) DNA from different species or to create genes with new functions. The resulting DNA copies are often referred to as recombinant DNA. Such recombinant DNA can subsequently be propagated in a host cell, such as without limitation a bacterial or yeast cell, whose cellular machinery copies and expresses the recombinant DNA along with its own. Recombinant DNA technology can be used to prepare chemicals or biomolecules that are otherwise challenging to isolate, synthesize, or obtain, at a larger scale and/or with a reduced cost.

In some embodiments, a recombinant DNA encoding the recombinant H-ferritin homopolymer may include or be included in a plasmid. As used herein, a “plasmid” is a circular, double-stranded DNA molecule. Plasmids are distinct from a cell's chromosomal DNA and are capable of autonomous replication. Plasmids may be used as vectors for insertion, expression, and propagation of foreign genes within a host organism. Such vectors may include specific sequences for an origin of replication, selectable markers, and cloning sites, enabling manipulation and study of genetic material for applications in research, biotechnology, and therapeutic development. As a nonlimiting example, a plasmid can include a nucleic acid sequence that encodes an H-ferritin subunit, e.g., with an amino acid sequence of SEQ ID NO: 1. As another nonlimiting example, a plasmid can include a pET30a(+) plasmid.

In some embodiments, the recombinant H-ferritin homopolymer may be expressed using host cells such as genetically modified BL21 cells, which is a strain of E. coli. As used herein, a “genetically modified” or “genetically engineered” cell is a cell whose genetic material has been manipulated to alter one or more of its hereditary traits. Such manipulation may include without limitation inserting, deleting, or otherwise modifying one or more specific DNA sequences; as a result of such manipulation, a genetically modified cell may exhibit one or more different traits in its structure, function, or the like, compared to its naturally occurring counterparts. As used herein, “BL21 cell” or “Escherichia coli BL21” is a commonly used protein production strain of Escherichia coli. This strain is derived from the B lineage of Escherichia coli and combines several features that allow for excessive expression of heterologous proteins. As used herein, a “heterologous” protein is a protein that is absent from a naturally occurring species. A heterologous protein may originate from a different species or be artificially designed. A heterologous protein may be expressed in a host cell using genetic engineering tools.

In some embodiments, the recombinant H-ferritin homopolymer is non-glycosylated and/or non-phosphorylated. Surprisingly, the use of a non-glycosylated and/or non-phosphorylated recombinant H-ferritin homopolymer in the composition provides a variety of benefits, such as without limitation improved iron supplementation, tissue iron distribution, and chemotherapy drug transport.

Transferrin is used as a benchmark for evaluating the biological functions of the composition and/or the recombinant H-ferritin homopolymer described herein. As used herein, “transferrin” is a blood plasma glycoprotein that plays a central role in iron metabolism and is responsible for ferric-ion delivery. Transferrin functions as a ferric pool in the body and transports iron through the blood to various tissues, such as the liver, spleen, and bone marrow. Transferrin is a free peptide (i.e., apo-transferrin) that undergoes a conformation change after binding with iron and can bind to two equivalents of ferric iron (Fe³⁺) with high affinity.

In some embodiments, the composition further includes at least one buffer solution, wherein the recombinant H-ferritin homopolymer is dissolved in the at least one buffer solution. As used herein, a “buffer” or “buffer solution” is a solution or mixture that contains at least one pair of weak acid, HA, and its conjugate base, A⁻, in a molar ratio typically between 10:1 and 1:10, wherein the solution or mixture maintains a stable pH close to the pKₐ of the weak acid, against addition of acidic or basic chemical species. For simplicity, a buffer containing a pair of conjugate base and acid can be written as A⁻/HA. As used herein, the “conjugate base” of a weak acid is a chemical species that contains exactly one fewer proton with respect to the weak acid. As used herein, the “pKₐ” of a weak acid is the negative log of the acid dissociation constant, Kₐ, of the weak acid. The pH of a buffer solution can be calculated using the Henderson Hasselbalch equation:

$$p H = p {K}_{a} + log ( \frac{[ {A}^{-} ]}{[ H A ]} ) .$$

A buffer can provide a stable chemical environment for the composition and/or the recombinant H-ferritin homopolymer therein and prevent it from denaturing. In some embodiments, the composition has, or is maintained at, a pH ranging from about 5.0 to about 7.0. In some embodiments, the composition has, or is maintained at, a pH ranging from about 6.0 to about 7.0. As nonlimiting examples, the composition can be maintained at a pH of about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, or about 7.0.

Any buffer solution known in the art to have either no/substantially no interaction or low interaction with iron metal ions can be used in the composition. As a nonlimiting example, a buffer can include an acetate buffer (i.e., CH₃COONa/CH₃COOH). As another nonlimiting example, a buffer can include a borate buffer (i.e., Na₂B₄O₇·10H₂O/H₃BO₃). As another nonlimiting example, a buffer can include a bicarbonate buffer (i.e., NaHCO₃/H₂CO₃ or Na₂CO₃/NaHCO₃, depending on the desired pH). As another nonlimiting example, a buffer can include a cacodylate buffer (i.e., NaC₂H₆AsO₂/HC₂H₆AsO₂). As a nonlimiting example, a buffer can include phosphate buffer (i.e., NaH₂PO₄/H₃PO₄, Na₂HPO₄/NaH₂PO₄, or Na₃HPO₄/Na₂HPO₄, depending on the desired pH).

As another nonlimiting example, a buffer can include a Good's buffer. As used herein, “Good's buffers” are a group of more than 20 conjugate acid/base pairs selected and described by Norman Good and colleagues for biochemical and biological research primarily during 1966-1980. For simplicity, only the conjugate acid may be shown for each conjugate acid/base pair. Nonlimiting examples of Good's buffers include MES (C₆H₁₃NO₄S), ADA (C₆H₁₀N₂O₅), PIPES (C₈H₁₈N₂O₆S₂), ACES (C₄H₁₀N₂O₄S), MOPSO (C₇H₁₅NO₅S), cholamine chloride hydrochloride (C₅H₁₆Cl₂N₂), MOPS (C₇H₁₅NO₄S), BES (C₆H₁₅NO₅S), TES (C₆H₁₅NO₆S), HEPES (C₈H₁₈N₂O₄S), TAPSO (C₇H₁₇NO₇S), POPSO (C₁₀H₂₂N₂O₈S₂), HEPPSO (C₉H₂₀N₂O₅S), EPPS or HEPPS (C₉H₂₀N₂O₄S), Tricine (C₆H₁₃NO₅), Tris (C₄H₁₁NO₃), glycinamide (C₂H₆N₂O), glycylglycine or Gly-Gly (C₄H₈N₂O₃), Bicine (C₆H₁₃NO₄), TAPS (C₇H₁₇NO₆S), AMPSO (C₇H₁₇NO₅S), CABS (C₁₀H₂₁NO₃S), CHES (C₈H₁₇NO₃S), CAPS (C₉H₁₉NO₃S), and CAPSO (C₉H₁₉NO₄S).

As another nonlimiting example, a buffer can include a phosphate-buffered saline (PBS) solution. As used herein, a “phosphate-buffered saline” or “PBS” is a commonly used buffer in biological research and pharmaceutical formulations that typically contains 137 mM NaCl, 2.7 mM KCl, 10 mM Na₂HPO₄, and 1.8 mM KH₂PO₄. As another nonlimiting example, a buffer can include an ethanesulfonic acid solution. As used herein, an “ethanesulfonic acid” solution is a buffer solution that contains one or more ethanesulfonic acid derivatives. An ethanesulfonic acid solution can be a solution of chemical(s) such as HEPES, consistent with details described above. As used herein, “HEPES”, also known as 4-(2-hydroxyethyl)piperazine-1-ethanesulfonic acid or by its IUPAC name as 2-[4-(2-hydroxyethyl)piperazin-1-yl]ethane-1-sulfonic acid, is a derivative of ethanesulfonic acid, with a formula of C₈H₁₈N₂O₄S, that contains a piperazine ring, —N(CH₂CH₂)₂N—. HEPES is a zwitterionic sulfonic acid buffering agent. As another nonlimiting example, a buffer can include a morpholino-based solution. As used herein, a “morpholino-based” solution is a buffer solution that includes one or more chemical species that contain a morpholine ring, —N(CH₂CH₂)₂O. A morpholino-based solution can be a solution of chemical(s) such as MOPS, consistent with details described above. As used herein, “MOPS”, also known as 3-(N-morpholino)propanesulfonic acid or by its IUPAC name as 3-(morpholin-4-yl)propane-1-sulfonic acid, is a chemical species with a formula of C₇H₁₅NO₄S.

It is worth noting that the classification of buffers described herein may, in some cases, be arbitrary and overlapping, and some buffers may accordingly be categorized under multiple categories.

In some embodiments, the composition and/or recombinant H-ferritin homopolymer includes a cavity. As used herein, a “cavity” is an empty, at least partially enclosed space or region in the recombinant H-ferritin homopolymer to which another chemical species may bind and be attached thereto or stored therein. In some embodiments, the cavity may have a diameter between about 2 nm and about 20 nm. As nonlimiting examples, the cavity may have a diameter of about 2 nm, about 3 nm, about 4 nm, about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, about 20 nm, and/or the like. In some embodiments, the cavity may contain a therapeutically active chemical species, such as without limitation a Fe²⁺ source, an iron element including Fe³⁺, and/or the like, consistent with details described elsewhere in this disclosure.

In some embodiments, the composition can be formulated for oral administration or delivery, consistent with details described elsewhere in this disclosure. In some embodiments, the composition can be formulated for intravenous administration or delivery, consistent with details described elsewhere in this disclosure. In some embodiments, the composition can be formulated for intraperitoneal administration or delivery, consistent with details described elsewhere in this disclosure.

In some embodiments, the composition further includes a saline solution. As used herein, a “saline” or “saline solution” is an aqueous solution of NaCl at a concentration of 0.9% w/w. A saline or saline solution can also be referred to as normal saline or isotonic saline.

In some embodiments, the composition further includes an antibacterial agent. As used herein, an “antibacterial agent” is a chemical species that kills bacteria or inhibits their growth. An antibacterial agent can include an antibiotic, including without limitation penicillins such as penicillin, amoxicillin, and ampicillin; cephalosporins such as cephalexin, cefazolin, cefdinir, cefaclor, cefepime, and ceftaroline; macrolides such as azithromycin, erythromycin, and clarithromycin; tetracyclines such as tetracycline, doxycycline, and minocycline; beta-lactams with increased activity such as amoxicillin/clavulanate and ceftazidime/avibactam; quinolones such as ciprofloxacin and levofloxacin; aminoglycosides such as streptomycin, gentamicin, and tobramycin, lincosamides such as clindamycin; and nitroimidazoles such as metronidazole and tinidazole. Additional examples of antibacterial agents include, but are not limited to, macrolides or ketolides such as erythromycin, azithromycin, clarithromycin, and telithromycin; beta-lactams including penicillin, cephalosporin, and carbapenems such as carbapenem, imipenem, and meropenem; monolactams such as penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, temocillin, cephalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, ceftriaxone, cefoperazone, ceftazidime, cefixime, cefpodoxime, ceftibuten, cefdinir, cefpirome, cefepime, and aztreonam; quinolones such as nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, ganefloxacin, gemifloxacin, and pazufloxacin; antibacterial sulfonamides and antibacterial sulphanilamides, such as para-aminobenzoic acid, sulfadiazine, sulfisoxazole, sulfamethoxazole, and sulfathalidine; aminoglycosides such as streptomycin, neomycin, kanamycin, paromycin, gentamicin, tobramycin, amikacin, netilmicin, spectinomycin, sisomicin, dibekalin, and isepamicin; tetracyclines such as tetracycline, chlortetracycline, demeclocycline, minocycline, oxytetracycline, methacycline, and doxycycline; rifamycins such as rifampicin (also called rifampin), rifapentine, rifabutin, bezoxazinorifamycin, and rifaximin; lincosamides such as lincomycin and clindamycin; glycopeptides such as vancomycin and teicoplanin; streptogramins such as quinupristin and daflopristin; oxazolidinones such as linezolid; polymyxin, colistin, and colymycin; trimethoprim, bacitracin, and phosphonomycin; fluoro-quinolones such as besifloxacin, clinafloxacin, garenoxacin, gemifloxacin, moxifloxacin, gatifloxacin, sitafloxacin, trovafloxacin, triclosan, alatrofloxacin, and prulifloxacin.

In some embodiments, the composition further includes an antioxidant. As used herein, an “antioxidant” is a chemical species that is capable of donating or transferring one or more of its electrons to an oxidant. By sacrificing itself and converting to its oxidation product, an antioxidant may prevent an oxidant from causing damages to biomolecules, such as without limitation the recombinant H-ferritin homopolymer or chemical species enclosed therein, or tissues of a subject to whom the composition is administered. Examples of antioxidants include, but are not limited to, carotenoids, tertiary butyl hydroquinone (TBHQ), butylated hydroxyanisole (BHA), butylated hydroxytoluene (BHT), vitamin E, vitamin C, and combinations thereof.

In some embodiments, the composition further includes a tonicity-adjusting agent. As used herein, a “tonicity-adjusting agent”, also known as a “tonicity modifier”, is a chemical species used to adjust the osmotic pressure of a solution, mixture, or composition to be isotonic with body fluids. The use of a tonicity-adjusting agent helps ensure the structural integrity and/or proper functioning of cells when exposed to the composition, preventing them from swelling under hypotonic conditions or shrinking under hypertonic conditions. Examples of tonicity adjusting agents include, but are not limited to, dextrose, glycerin, mannitol, potassium chloride, and sodium chloride.

In some embodiments, the composition includes a Fe²⁺ source. As used herein, a “Fe²⁺ source” is a chemical species capable of providing or releasing Fe²⁺ upon administration of the composition to a subject. The Fe²⁺ source can be selected from, but is not limited to, ferrous chloride, ferrous nitrate, ferrous oxide, ferrous bromide, iron(II) carbonate, ferrous sulfate, ferrous sulfide, and ferrous ammonium sulfate.

Another aspect of the present invention is a genetically engineered microbe or host cell that produces the recombinant H-ferritin homopolymer described herein. In some embodiments, the genetically engineered microbe or host cell is a genetically engineered E. coli strain prepared using recombinant DNA technology. As a nonlimiting example, such genetically engineered E. coli strain can be a BL21 strain. In some embodiments, the genetically engineered microbe or host cell can be prepared by transfecting a wild-type strain using a plasmid, such as pET30a(+) plasmid, wherein the plasmid includes a nucleic acid sequence encoding an H-ferritin subunit. The H-ferritin subunit can have an amino acid sequence of SEQ ID NO: 1. As another nonlimiting example, a plurality (e.g., 24) of H-ferritin subunits can undergo a spontaneous (i.e., organic) self-assembly process to form the H-ferritin homopolymer; this process can occur in the cytosol of the genetically engineered microbe or host cell.

Another aspect of the present disclosure is a method for treating an iron-related condition in a subject, the method including administering any composition disclosed herein that includes a holo-recombinant H-ferritin homopolymer, wherein the holo-recombinant H-ferritin homopolymer has a plurality of H-ferritin subunits.

In some embodiments, the holo-recombinant H-ferritin homopolymer used in the method includes at least 24 H-ferritin subunits.

In some embodiments, at least one H-ferritin subunit of the plurality of H-ferritin subunits has an amino acid sequence of SEQ ID NO: 1.

The holo-recombinant H-ferritin homopolymer stores an iron element. As used herein, an “iron element” is a chemical species, or a group of chemical species, that contains the element Fe. In some embodiments, the iron element stored in the holo-recombinant H-ferritin homopolymer is a ferric iron (i.e., Fe³⁺) or a salt, complex, or compound thereof.

As used herein, an “iron-related condition” is a condition resulting from or associated with an excess, deficiency, or dysregulation of iron species. In some embodiments, an iron-related condition can be a condition such as, but not limited to, restless legs syndrome (RLS), attention-deficit/hyperactivity disorder (ADHD), Parkinson's Disease, and/or iron deficiency anemia. As used herein, “restless legs syndrome” or “RLS”, also known as Willis-Ekbom Disease, is a neurological condition that causes an irresistible urge to move the legs. RLS is a sleep disorder triggered by resting and attempting to sleep. RLS is also a movement condition, as people with RLS are forced to move their legs to find relief from symptoms. People with RLS generally feel an irresistible urge to move, accompanied by uncomfortable sensations in their legs. The sensations may feel like aching, throbbing, pulling, itching, crawling, or creeping. RLS less commonly affects the arms, rarely occurs in the chest or head, and most often affects both sides of the body. As used herein, an “attention-deficit/hyperactivity disorder” or “ADHD” is a developmental disorder characterized by an ongoing pattern of one or more of the following types of symptoms: i) inattention, such as having difficulty paying attention, keeping on task, or staying organized; ii) hyperactivity, such as often moving around (including during inappropriate times), feeling restless, or talking excessively; and (iii) impulsivity, such as interrupting, intruding on others, or having trouble waiting one's turn. As used herein, “Parkinson's Disease” or “PD” is a neurodegenerative disease primarily of the central nervous system, affecting both motor and non-motor systems. Symptoms of PD typically develop gradually, with non-motor issues becoming more prevalent as the disease progresses. The motor symptoms are collectively called parkinsonism and include tremors, bradykinesia, rigidity, as well as postural instability (i.e., difficulty maintaining balance). Non-motor symptoms develop later in the disease and include behavioral changes or neuropsychiatric problems such as sleep abnormalities, psychosis, and mood swings. As used herein, “iron deficiency anemia” is a type of anemia that develops due to the lack of iron. Iron deficiency anemia is the most common cause of anemia worldwide, which results in microcytic and hypochromic red cells on the peripheral smear. The cause of iron-deficiency anemia can vary based on age, gender, and socioeconomic status of the subject. Iron deficiency may result from insufficient iron intake, decreased absorption, or blood loss. Iron deficiency anemia is most often from blood loss, especially in older subjects. Iron deficiency anemia can also be seen with low dietary intake, increased systemic requirements for iron such as in pregnancy, and decreased iron absorption such as in celiac disease. As used herein, “anemia” is a condition where the level of hemoglobin in a subject is at least two standard deviations below the mean for the age and gender of the subject.

As used herein, a “subject” includes, but is not limited to, humans and non-human vertebrates such as wild, domestic, and farm animals. The terms “subject” and “patient” may be used interchangeably throughout this disclosure. In some embodiments, a subject can be a mammal (e.g., a dog or a pig). In some embodiments, a subject can be a human. As a nonlimiting example, a subject or patient may include a human of any age, sex, gender, race, ethnicity, health record, etc., as deemed relevant and/or suitable by a person of ordinary skill in the art, upon reviewing the entirety of this disclosure.

In some embodiments, the composition used in the method includes about 10 mg to about 1,000 mg of the iron element. As nonlimiting examples, the composition can include from 10 mg to 20 mg, from 20 mg to 30 mg, from 30 to 40 mg, from 40 mg to 50 mg, from 50 mg to 60 mg, from 60 mg to 70 mg, from 70 mg to 80 mg, from 80 mg to 90 mg, from 90 mg to 100 mg, from 100 mg to 200 mg, from 200 mg to 300 mg, from 300 mg to 400 mg, from 400 mg to 500 mg, from 500 mg to 600 mg, from 600 mg to 700 mg, from 700 mg to 800 mg, from 800 mg to 900 mg, from 900 mg to 1,000 mg, and/or the like. As further nonlimiting examples, the composition can include about 10 mg, about 20 mg, about 30 mg, about 40 mg, about 50 mg, about 60 mg, about 70 mg, about 80 mg, about 90 mg, about 100 mg, about 200 mg, about 300 mg, about 400 mg, about 500 mg, about 600 mg, about 700 mg, about 800 mg, about 900 mg, about 1,000 mg, and/or the like, of the iron element.

In some embodiments, the composition used in the method is orally, intravenously, or intraperitoneally administered to the subject, in an amount from about 3.4 mg/kg of body weight to about 25 mg/kg of body weight. As nonlimiting examples, the composition used in the method may be in an amount from about 3.4 mg/kg of body weight to about 4 mg/kg of body weight, from about 4 mg/kg of body weight to about 5 mg/kg of body weight, from about 5 mg/kg of body weight to about 6 mg/kg of body weight, from about 6 mg/kg of body weight to about 7 mg/kg of body weight, from about 7 mg/kg of body weight to about 8 mg/kg of body weight, from about 8 mg/kg of body weight to about 9 mg/kg of body weight, from about 9 mg/kg of body weight to about 10 mg/kg of body weight, from about 10 mg/kg of body weight to about 12 mg/kg of body weight, from about 12 mg/kg of body weight to about 14 mg/kg of body weight, from about 14 mg/kg of body weight to about 16 mg/kg of body weight, from about 16 mg/kg of body weight to about 18 mg/kg of body weight, from about 18 mg/kg of body weight to about 20 mg/kg of body weight, from about 20 mg/kg of body weight to about 24 mg/kg of body weight, from about 24 mg/kg of body weight to about 25 mg/kg of body weight, and/or the like. As further nonlimiting examples, the composition used in the method may be in an amount of about 3.4 mg/kg of body weight, about 4 mg/kg of body weight, about 5 mg/kg of body weight, about 6 mg/kg of body weight, about 7 mg/kg of body weight, about 8 mg/kg of body weight, about 9 mg/kg of body weight, about 10 mg/kg of body weight, about 12 mg/kg of body weight, about 14 mg/kg of body weight, about 16 mg/kg of body weight, about 18 mg/kg of body weight, about 20 mg/kg of body weight, about 22 mg/kg of body weight, about 24 mg/kg of body weight, about 25 mg/kg of body weight, and/or the like.

Another aspect of the present disclosure is a method for treating a neurological or blood disorder in a subject, the method including administering any composition disclosed herein that includes a recombinant H-ferritin homopolymer having a plurality of H-ferritin subunits.

In some embodiments, the recombinant H-ferritin homopolymer used in the method includes at least 24 H-ferritin subunits.

In some embodiments, at least one H-ferritin subunit of the plurality of H-ferritin subunits has an amino acid sequence of SEQ ID NO: 1.

In some embodiments, the composition used in the method includes a Fe²⁺ source. Nonlimiting examples of Fe²⁺ sources include without limitation ferrous chloride, ferrous nitrate, ferrous oxide, ferrous bromide, iron(II) carbonate, ferrous sulfate, ferrous sulfide, and/or ferrous ammonium sulfate, consistent with details described elsewhere in this disclosure.

In some embodiments, the composition used in the method includes about 10 mg to about 1,000 mg of the recombinant H-ferritin homopolymer. As nonlimiting examples, the composition can include from 10 mg to 20 mg, from 20 mg to 30 mg, from 30 to 40 mg, from 40 mg to 50 mg, from 50 mg to 60 mg, from 60 mg to 70 mg, from 70 mg to 80 mg, from 80 mg to 90 mg, from 90 mg to 100 mg, from 100 mg to 200 mg, from 200 mg to 300 mg, from 300 mg to 400 mg, from 400 mg to 500 mg, from 500 mg to 600 mg, from 600 mg to 700 mg, from 700 mg to 800 mg, from 800 mg to 900 mg, from 900 mg to 1,000 mg, and/or the like. As further nonlimiting examples, the composition can include about 10 mg, about 20 mg, about 30 mg, about 40 mg, about 50 mg, about 60 mg, about 70 mg, about 80 mg, about 90 mg, about 100 mg, about 200 mg, about 300 mg, about 400 mg, about 500 mg, about 600 mg, about 700 mg, about 800 mg, about 900 mg, about 1,000 mg, and/or the like, of the recombinant H-ferritin homopolymer.

In some embodiments, the composition used in the method can be orally, intravenously, or intraperitoneally administered to the subject, in an amount from about 3.4 mg/kg of body weight to about 25 mg/kg of body weight. As nonlimiting examples, the composition used in the method may be in an amount from about 3.4 mg/kg of body weight to about 4 mg/kg of body weight, from about 4 mg/kg of body weight to about 5 mg/kg of body weight, from about 5 mg/kg of body weight to about 6 mg/kg of body weight, from about 6 mg/kg of body weight to about 7 mg/kg of body weight, from about 7 mg/kg of body weight to about 8 mg/kg of body weight, from about 8 mg/kg of body weight to about 9 mg/kg of body weight, from about 9 mg/kg of body weight to about 10 mg/kg of body weight, from about 10 mg/kg of body weight to about 12 mg/kg of body weight, from about 12 mg/kg of body weight to about 14 mg/kg of body weight, from about 14 mg/kg of body weight to about 16 mg/kg of body weight, from about 16 mg/kg of body weight to about 18 mg/kg of body weight, from about 18 mg/kg of body weight to about 20 mg/kg of body weight, from about 20 mg/kg of body weight to about 24 mg/kg of body weight, from about 24 mg/kg of body weight to about 25 mg/kg of body weight, and/or the like. As further nonlimiting examples, the composition used in the method may be in an amount of about 3.4 mg/kg of body weight, about 4 mg/kg of body weight, about 5 mg/kg of body weight, about 6 mg/kg of body weight, about 7 mg/kg of body weight, about 8 mg/kg of body weight, about 9 mg/kg of body weight, about 10 mg/kg of body weight, about 12 mg/kg of body weight, about 14 mg/kg of body weight, about 16 mg/kg of body weight, about 18 mg/kg of body weight, about 20 mg/kg of body weight, about 22 mg/kg of body weight, about 24 mg/kg of body weight, about 25 mg/kg of body weight, and/or the like.

In some embodiments, the method is used to treat a neurological or blood disorder such as without limitation hemochromatosis, β-thalassemia, Parkinson's disease, stroke, and/or an iron overload condition. As used herein, “hemochromatosis” is a disorder in which extra iron builds up in the body to harmful levels. Without treatment, hemochromatosis can cause iron overload, a buildup of iron that can damage certain parts of the body, including without limitation the liver, heart, pancreas, endocrine glands, and joints. As used herein, “β-thalassemia” is an inherited blood disorder characterized by reduced levels of functional hemoglobin. A characteristic finding of β-thalassemia is anemia, due to red blood cells being abnormally small (microcytic), not being produced at normal amounts, and/or not containing enough functional hemoglobin. Consequently, individuals affected by β-thalassemia do not receive enough oxygen-rich blood throughout the body and may subsequently experience classic symptoms of anemia including fatigue, weakness, shortness of breath, dizziness, or headaches. As used herein, a “stroke”, also known as a brain attack or cerebrovascular accident (CVA), is a condition where blood flow to the brain is interrupted or reduced, which causes brain tissue damage. As used herein, an “iron overload condition” is a medical condition caused by an oversupply of iron in the body. Excess iron can be toxic, as unregulated free iron can catalyze the production of harmful reactive oxygen species (ROS) and free radicals, most commonly via Fenton chemistry. Such species are strong oxidizing agents that can cause permanent cell damage, organ failure, and/or the like.

### EXAMPLES

### Example 1

### Methods

### Animal Experiment—C57BL/6J Mice and Diets

Male and female C57BL/6J mice were procured from the Jackson laboratory. Diet-induced ID was performed as described previously (see Unger E L, Hurst A R, Georgieff M K, et al., “Behavior and mono-amine deficits in prenatal and perinatal iron deficiency are not corrected by early postnatal moderate-iron or high-iron diets in rats”, J. Nutr., 2012; 142(11):2040-2049). Briefly, female breeders were fed a 35 mg/kg iron diet (AIN93G-control diet) for seven days. This diet was found to be adequate for pregnancy outcomes but does not allow for increased iron stores in the dam. Afterward, mice mated, and pregnancy was confirmed by the presence of a vaginal plug. At gestational (G) day 5, the pregnant dams either continued on the control diet for the control group (iron 35 mg/kg diet, AIN-93G), or were switched to an iron-deficient diet for the ID group (iron 3.5 mg/kg diet #AIN-93), until the pups reached postnatal day (PND) 15. At PND14, the mice were injected with either ⁵⁵Fe-transferrin (⁵⁵Fe-Tf) or an ⁵⁵Fe—H-ferritin recombinant homopolymer (⁵⁵Fe-FTH1) as disclosed herein. Tissues were harvested after 24 hours. For the adult ID model, pregnant dams and pups were fed with a control diet until PND45. At PND45, the mice were divided into two groups; a control group that continued on the same diet (iron 35 mg/kg diet, AIN-93G) and an ID group that was fed an ID diet (iron 3.5 mg/kg diet #AIN-93) until PND84. At PND84, mice were injected with ⁵⁵Fe-Tf or ⁵⁵Fe-FTH1 and tissues were harvested after 24 hours.

### His-H-ferritin

Recombinant FTH1 was prepared as previously described (see Bruner A B, Joffe A, Duggan A K, Casella J F, Brandt J., “Randomised study of cognitive effects of iron supplementation in non-anemic iron-deficient adolescent girls”, Lancet, 1996; 348(9033):992-996). Briefly, wild-type human FTH1 containing a poly-His tag was subcloned into pET30a (+), to be produced in BL21 Escherichia coli. Isopropyl-β-D-thio-galactoside (IPTG) was used to induce expression. Following this step, bacteria were lysed, and FTH1 protein was purified on a nickel column using standard techniques (GE Healthcare Bio-Sciences)

### ⁵⁵Fe-Labeling of FTH1 or Tf

The ⁵⁵Fe labeling of Tf or FTH1 was performed as previously described (see Bruner A B, Joffe A, Duggan A K, Casella J F, Brandt J., “Randomised study of cognitive effects of iron supplementation in non-anaemic iron-deficient adolescent girls”, Lancet, 1996; 348(9033):992-996). Briefly, ⁵⁵Fe (Perkin Elmer) was complexed with nitrilotriacetic acid (NTA), ferric chloride (FeCl₃), and sodium bicarbonate (NaHCO₃) at a ratio of 100 μL NTA:6.7 μL FeCl₃:23.3 μL NaHCO₃:50 μCi ⁵⁵FeCl₃ to form the ⁵⁵Fe-NTA complex. After complexing, ⁵⁵Fe-NTA was subsequently incubated with apo-Tf (Sigma) or His-FTH1 for 30 min to allow for iron loading (see Knutson M D, “Iron transport proteins: gateways of cellular and systemic iron homeostasis”, J. Biol Chem., 2017; 292(31):12735-12743). Free iron was separated from the total complex using PD midiTrap-G25 columns following the manufacturer's instructions (GE Healthcare Bio-Sciences.

### ⁵⁵Fe Uptake Studies

Mice received a single intraperitoneal injection of 3.4 mg/kg body weight ⁵⁵Fe-Tf or ⁵⁵Fe-FTH1 with 50 μCi of ⁵⁵FeCl₃. Twenty-four hours after injection, blood was drawn, and mice were transcardially perfused with 0.1 M phosphate-buffered saline (PBS, pH 7.4). Brains were collected, weighed immediately, and solubilized using 1 mL Solvable (Perkin Elmer) according to the manufacturer's instructions. After solubilization, a 10 mL Hionic-Fluor scintillation cocktail (Perkin Elmer) was added. Samples were counted using the Hidex 300 SL (LabLogic) for three minutes each. Blank tube values were subtracted from the final counts to correct for background counts.

### Statistics

All the data were presented as the mean±SD. Statistical analysis was employed with the GraphPad Prism version 9.0 software. Comparison of differences between two or multiple groups by Student's t-test and two-way ANOVA respectively. p<0.05 was considered statistically significant.

### Results

Developmental ID is Associated with Increased Brain Uptake of ⁵⁵Fe-FTH1 and ⁵⁵Fe-Tf in Male and Female Mice.

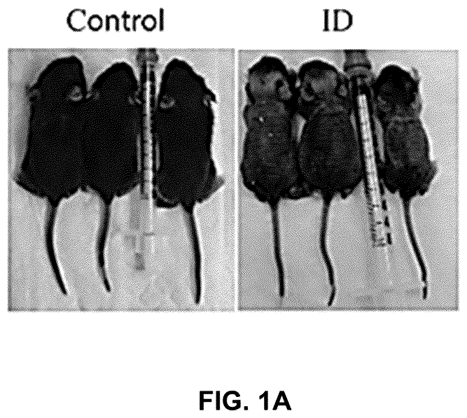

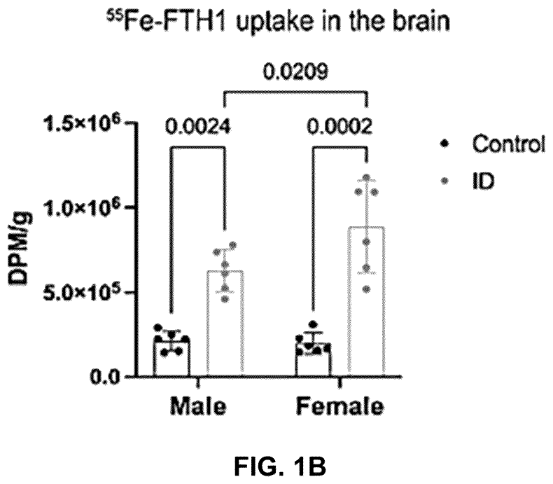

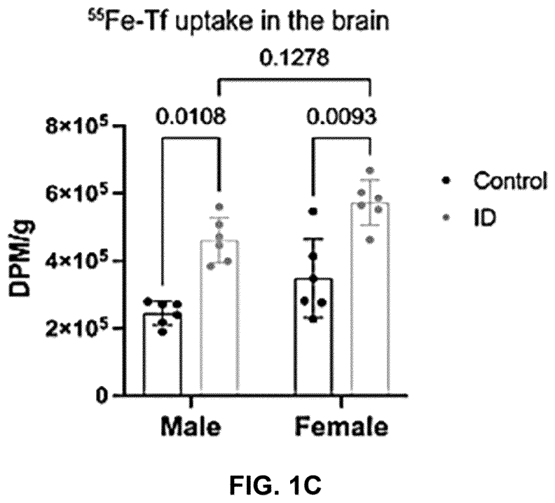

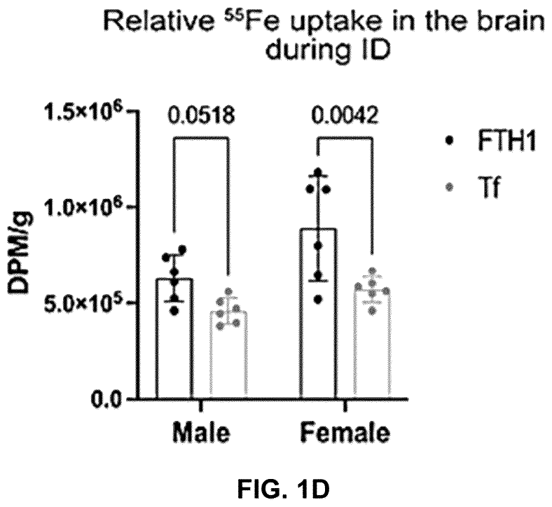

In the developmental model (i.e., mice at PND 15), ID was confirmed by differences in weight, color, and amount of hair between the two groups (see FIG. 1A). Subsequently, iron uptake was measured in the brain after ⁵⁵Fe-FTH1 or ⁵⁵Fe-Tf treatment. To measure iron uptake into the brain, 3.5 mg/kg of FTH1 or Tf with 50 μCi of ⁵⁵Fe was loaded and injected intraperitoneally into the developing mice. After 24 hours, the animals were perfused with 0.1 M PBS, and brains were collected to measure iron uptake.

Brain uptake of ⁵⁵Fe-FTH1 and ⁵⁵Fe-Tf increased significantly in the ID group in both sexes compared to corresponding control male and female mice (see FIGS. 1B-C). The interaction between the diet and sex was also measured, wherein a significant difference in the uptake of ⁵⁵Fe-FTH1 in males and females was detected (see FIG. 1B). There was no statistically significant increase in the uptake of ⁵⁵Fe-Tf in female mice compared to the males (see FIG. 1C). The difference between the amount of iron delivered by each of the proteins was also measured. More ⁵⁵Fe was found to be taken into the brain under ID condition by ⁵⁵Fe-FTH1 than ⁵⁵Fe-Tf in both sexes (see FIG. 1D).

The Uptake of ⁵⁵Fe-FTH1 and ⁵⁵Fe-Tf into the Brains of Male and Female Mice are Different in the Adult ID Model.

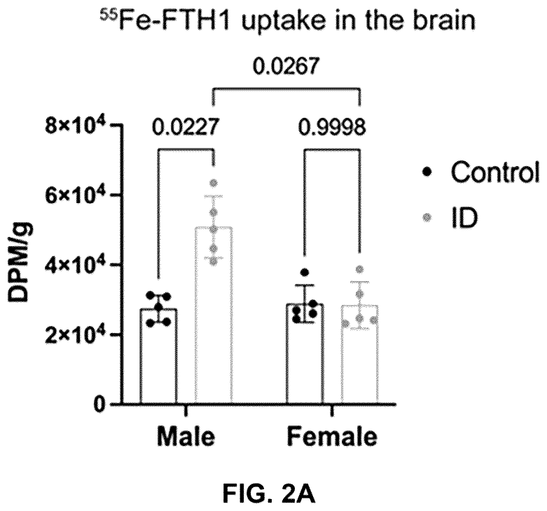

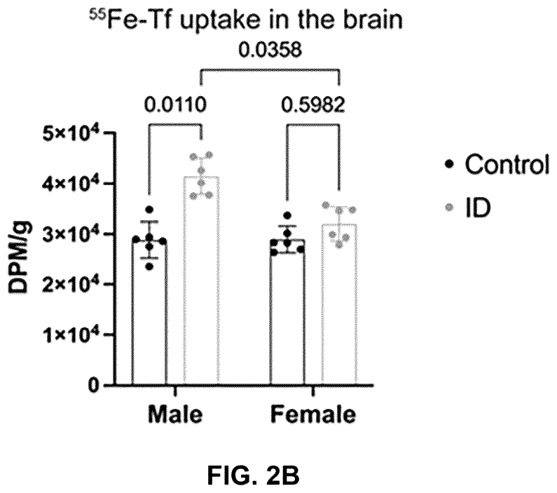

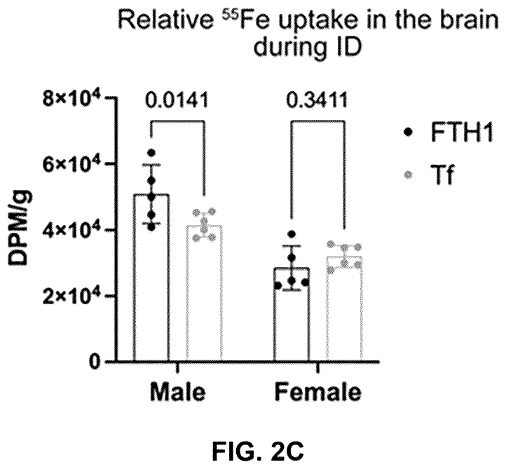

Following confirmation of ID in adult mice, the ⁵⁵Fe uptake into the brains of ID adult mice was examined. In the adult model of ID, ⁵⁵Fe-FTH1 and ⁵⁵Fe-Tf uptake into the brain was significantly higher in the ID group compared to the control group in the male mice brains, but there are no ID-induced differences in the ⁵⁵Fe-FTH1 or ⁵⁵Fe-Tf uptake in brains of the female mice (see FIGS. 2A-B).

Sex-dependent differences in ⁵⁵Fe uptake during ID was also measured. The ⁵⁵Fe uptake from both Tf and FTH1 was greater in ID males compared to females (see FIG. 2C). Moreover, the FTH1-bound iron uptake was significantly higher than the Tf-bound iron uptake in males, whereas in females, there is no difference between the iron delivery from either protein (see FIG. 2C).

The Uptake of ⁵⁵Fe-FTH1 and ⁵⁵Fe-Tf was Greater in Developing Mice Brains Compared to Adult Mice Brains.

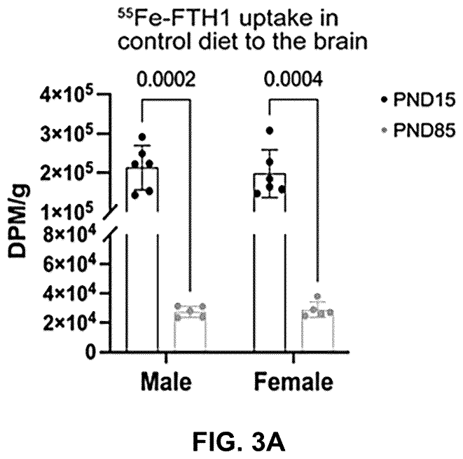

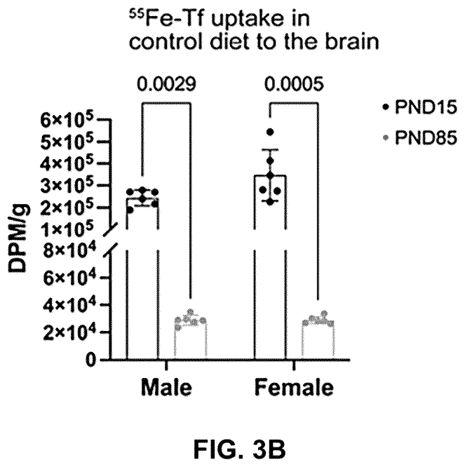

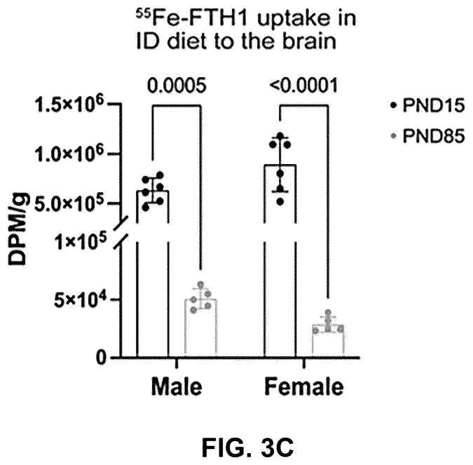

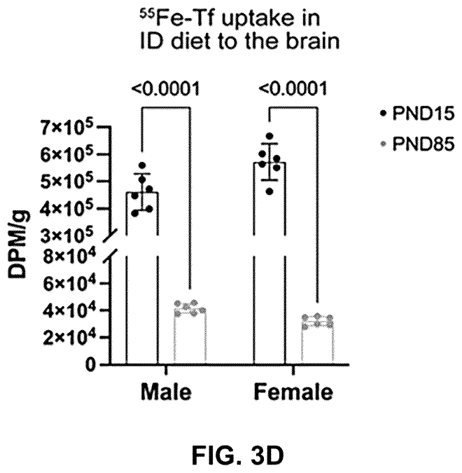

The uptake of ⁵⁵Fe-FTH1 and ⁵⁵Fe-Tf was greater in developing mice brains compared to adult mice brains. Following this discovery, the effect that diet has on FTH1- and Tf-bound iron uptake were explored. First, the ⁵⁵Fe uptake between developing and adult brains on a control diet was measured. The ⁵⁵Fe uptake was significantly higher in the developing group compared to the adults for both FTH1 and Tf (see FIGS. 3A-B). Next, the ⁵⁵Fe uptake between developing and adult brains on an ID diet was measured. The FTH1- and Tf-bound ⁵⁵Fe uptake was 10 times higher in the developing mice (PND15) compared to the adult mice (PND85) (see FIGS. 3A-D).

### Example 2

In this example, the role of recombinant apo-FTH1 (homopolymer) on iron redistribution in Tfhpx/hpx mice was examined. Trfhpx/hpx mice is a model of inherited deficiency of the serum iron-binding protein transferrin. These mice develop anemia because transferrin is essential for iron delivery to erythroid precursors. They also develop iron excess because transferrin is essential for hepcidin expression.

### Methods

### Animals and FTH1 Treatment

BALB/cJ Trf and Trfhpx/hpx mice were generated by crossing Trf+/hpx mice, which were intermittently backcrossed to BALB/cJ mice (Jackson Laboratories). To ensure survival of Trfhpx/hpx mice after weaning, pups were injected intraperitoneally with 3 mg human transferrin (Roche/Sigma) 2 days after birth, then once a week until weaning at 3 weeks of age. After weaning, hpx mice were injected intraperitoneally with 3 mg human transferrin three times a week. Before starting the experiment, mice were on the without transferrin injections for a week. Afterwards, 25 ug/g of Tf was injected into the transferrin group, 25 ug/g of recombinant apo-FTH1 (prepared from a BL21 E. coli strain that has been transfected using a pET30a(+) plasmid) was injected into the FTH1 group, and PBS was injected into the control group (HPX) for 7 days. After 24 hours from the last injection, the mice were euthanized, and tissues were collected for iron estimation.

### Recombinant Apo-H-Ferritin Preparation

Wild-type human FTH1 containing a poly-His tag was subcloned into pET30a(+), to be produced in BL21 Escherichia coli. Isopropyl-β-D-thio-galactoside (IPTG) was used to induce expression. Following this step, bacteria were lysed, and FTH1 protein was purified on a nickel column using standard techniques (GE Healthcare Bio-Sciences). Transferrin was purchased commercially (Sigma).

### Hematology

The hemoglobin and hematocrit levels were measured using a Heska HT5 Veterinarian Hematology Analyzer (Heska Antech Company, Loveland, CO, USA).

### Tissue Iron Estimation by Inductively Coupled Plasma-Atomic Emission Spectrometry (ICP-AES)

Tissue acid digestion was performed as described previously. Briefly, wet liver, spleen, and brains were digested overnight with nitric acid (2 mL) and, 30% hydrogen peroxide (1 mL) at 60° C. Iron concentration was determined by ICP-AES against a standard. Results were expressed as μg/g of wet tissue weight.

### Results

### Recombinant FTH1 Did not Increase Hb and HCT.

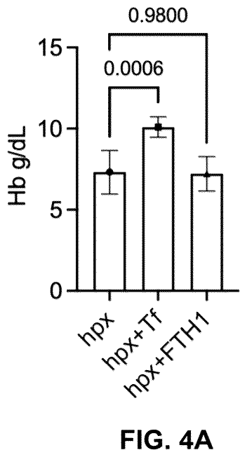

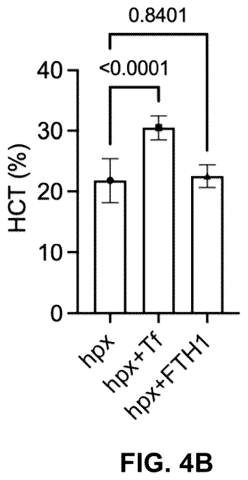

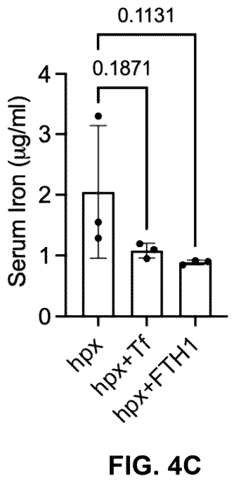

25 ug/g of Tf was injected into the transferrin group, 25 ug/g of recombinant apo-FTH1 was injected into the FTH1 group, and PBS was injected into the control group (HPX) for 7 days. After 7 days, blood was collected to measure levels of Hb, HCT, and serum iron. The Hb and HCT levels did not change in the FTH1-injected group compared to the control hpx group, whereas in the Tf-injected group, a significant increase in Hb and HCT level were detected when compared to the FTH1 and control groups (see FIGS. 4A-B). The serum iron concentration levels were also measured and found to have decreased in the FTH1- and Tf-injected groups when compared to the control (hpx) group (see FIG. 4C).

### Recombinant Apo-FTH1 Treatment Decreases the Excess Liver Iron in the hpx Mice.

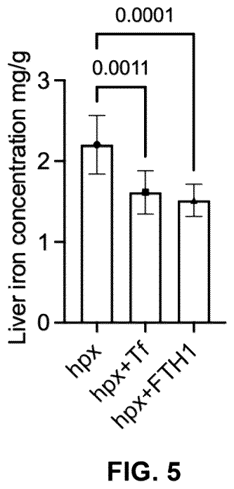

In hpx mice, iron levels were accumulated in the liver due to the absence of blood Tf and hepcidin. In this study, the liver iron levels in hpx mice after treatment with apo-FTH1 were measured. Apo-FTH1 treatment significantly decreased liver iron levels compared to the control group (see FIG. 5). The Apo-FTH1 homopolymer contained 24 subunits of FTH1, which has ferroxidase activity and stores 4500 iron atoms.

### Recombinant Apo-FTH1 Treatment Increases the Spleen Iron Levels in the hpx Mice.

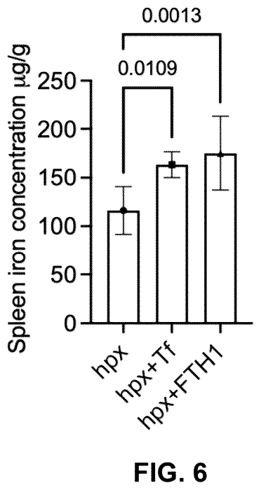

The spleen iron levels in the apo-FTH1 injected group were also measured. The apo-FTH1 treatment was discovered to significantly increase spleen iron levels when compared to the control hpx group (see FIG. 6).

### Recombinant Apo-FTH1 Treatment Did not Alter the Brain Iron Levels in the hpx Mice.

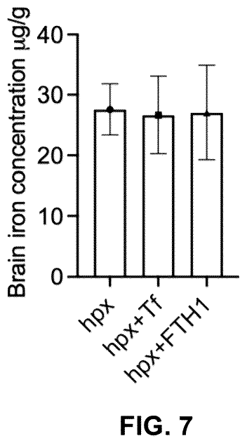

Brain iron concentration levels were also measured in hpx mice after FTH1 and Tf treatment. Apo-FTH1 and Tf injection did not change brain iron levels when compared to the hpx control group (see FIG. 7).

It will be apparent to those skilled in the art that numerous modifications and variations of the described examples and embodiments are possible in light of the above teachings of the disclosure. The disclosed examples and embodiments are presented for purposes of illustration only. Other alternative embodiments may include some or all of the features of the various embodiments disclosed herein. For instance, it is contemplated that a particular feature described, either individually or as part of an embodiment, can be combined with other individually described features, or parts of other embodiments. The elements and acts of the various embodiments described herein can therefore be combined to provide further embodiments.

It is the intent to cover all such modifications and alternative embodiments as may come within the true scope of this disclosure, which is to be given the full breadth thereof. Additionally, the disclosure of a range of values is a disclosure of every numerical value within that range, including the end points. Thus, while certain exemplary embodiments of the device and methods of making and using the same have been discussed and illustrated herein, it is to be distinctly understood that the disclosure is not limited thereto but may be otherwise variously embodied and practiced within the scope of the following claims.

1. A composition comprising:
a recombinant H-ferritin homopolymer comprising a plurality of H-ferritin subunits, wherein at least one H-ferritin subunit of the plurality of H-ferritin subunits has an amino acid sequence of SEQ ID NO: 1.

2. The composition according to claim 1, wherein the recombinant H-ferritin homopolymer has a molecular weight of approximately 500 kDa.

3. The composition according to claim 1, wherein the plurality of H-ferritin subunits comprises 24 H-ferritin subunits.

4. The composition according to claim 1, further comprising at least one buffer solution selected from a group consisting of a phosphate-buffered saline (PBS) solution, an ethanesulfonic acid solution, and a morpholino-based solution, wherein the recombinant H-ferritin homopolymer is dissolved in the at least one buffer solution.

5. The composition according to claim 1, wherein the composition has a pH ranging from about 5.0 to about 7.0.

6. The composition according to claim 1, further comprising one or more members selected from a group consisting of a saline solution, an antibacterial agent, an antioxidant, and a toxicity-adjusting agent.

7. The composition according to claim 1, further comprising a Fe²⁺ source.

8. The composition according to claim 1, wherein the recombinant H-ferritin homopolymer is a non-phosphorylated or non-glycosylated recombinant H-ferritin homopolymer.

9. A method for treating an iron-related condition in a subject, wherein the method comprises:
administering, to the subject with the iron-related condition, a therapeutically effective amount of composition comprising:
a holo-recombinant H-ferritin homopolymer; and
an iron element stored within the holo-recombinant H-ferritin homopolymer, wherein:
the holo-recombinant H-ferritin homopolymer comprises a plurality of H-ferritin subunits; and
at least one H-ferritin subunit of the plurality of H-ferritin subunits has an amino acid sequence of SEQ ID NO: 1.

10. The method according to claim 9, wherein the iron element includes Fe³⁺.

11. The method according to claim 9, wherein the subject is a mammal.

12. The method according to claim 11, wherein the subject is a human.

13. The method according to claim 9, wherein the composition comprises from 10 mg to 1,000 mg of the iron element.

14. The method of claim 9, wherein the iron-related condition is an iron deficiency condition.

15. A method for treating a neurological or blood disorder in a subject, wherein the method comprises administering, to the subject with the neurological or blood disorder, a therapeutically effective amount of a composition comprising:
a recombinant H-ferritin homopolymer comprising a plurality of H-ferritin subunits, wherein at least one H-ferritin subunit of the plurality of H-ferritin subunits has an amino acid sequence of SEQ ID NO: 1.

16. The method according to claim 15, wherein the composition further comprises a Fe²⁺ source.

17. The method according to claim 15, wherein the subject is a mammal.

18. The method according to claim 17, wherein the subject is a human.

19. The method of claim 15, wherein the composition comprises from 10 mg to 1,000 mg of the recombinant H-ferritin homopolymer.

20. The method according to claim 15, wherein the neurological or blood disorder is one or more members selected from a group consisting of hemochromatosis, β-thalassemia, Parkinson's disease, stroke, and an iron overload condition.


SEQUENCE LISTING (1 sequence)

SEQ ID NO: 1 - protein, 165 aa, synthetic construct [sequence omitted]